(19) United States
(12) Patent Application Publication
(10) Pub. No.: US 2024/0084238 A1
(43) Pub. Date: Mar. 14, 2024

(54) USE OF 3D POROUS STRUCTURE FOR PLATELET PRODUCTION

(71) Applicant: HEMOSTOD SA, Lausanne (CH)

(72) Inventors: Elodie Dahan, Lausanne (CH); Sophie Claudel, Lausanne (CH); Magali Humbert, Lausanne (CH)

(21) Appl. No.: 18/262,179

(22) PCT Filed: Feb. 8, 2022
(86) PCT No.: PCT/EP2022/052946

§ 371 Date: Jul. 19, 2023

(30) Foreign Application Priority Data
Feb. 8, 2021 (EP) 21155887.9

(51) Int. Cl. C12M 1/26; C12N 5/078

(52) U.S. Cl. CPC C12M 33/14; C12N 5/0644

(57) ABSTRACT

A method and device for large-scale platelet production in vitro are provided. The method uses a platelet production device comprising a rotatable bed reactor configured to contain a porous material for producing platelet from megakaryocytes at large scale.

### TECHNICAL FIELD

The present invention relates to platelet production in vitro. Specifically, the present invention discloses a method and device to produce platelets from megakaryocytes (MK) at large scale.

### TECHNICAL BACKGROUND

Platelets are small anucleate blood cells whose function is to stop bleeding. Patients who have low platelet counts (thrombocytopenia) due to certain diseases, treatments or after major hemorrhages, require platelet transfusion—a life-saving product. Currently, the only source is blood donation. Nevertheless, as platelets have a shelf-life of five days, hospital stocks need to be continuously refreshed and shortage issues are frequent (˜20%) in developed countries (epidemic, bad weather, vacation period . . . ), whereas more than half of the needs are unserved in emerging countries. Shortage issues may lead to mismatched transfusions that can result in inefficient transfusions or adverse events including alloimmunization. Any such events, in the fragile population of patients requiring platelet transfusions is a real hurdle to manage, associated with longer hospital stays, higher inpatient hospital costs and reduced survival. In-vitro platelet production for therapeutic applications is an appealing alternative but remains a major technological challenge, especially in terms of scalability of the process toward an industrial production.

Platelets originate from mature megakaryocytes. The mature megakaryocytes are the result of a process occurring in the bone marrow that involves the commitment of multipotent hematopoietic stem cells toward MK progenitors, the proliferation and differentiation of these progenitors, their polyploidization and their maturation. Through a dynamic process, cytoplasm of mature MK form long pseudopodial elongations (designated pro-platelets) through the vascular environment to release discoid platelets in the sinusoidal blood vessels. It has also been established that the “organ” of platelet production consists not only of the bone marrow, but also of the lungs where MK can produce these elongations directly in the lung microcirculation in a flow-dependent manner (Lefrancais et al.; “The lung is a site of platelet biogenesis and a reservoir for hematopoietic progenitors”; Nature. 2017 Apr. 6; 544(7648): 105-109). Numerous attempts of designing bioreactors dedicated to platelet production have been made.

Bioreactors based on a bone marrow environment model have generally two compartments: one where MK are seeded and the other where platelets are collected. The dynamic passage from the bone marrow (MK compartment) to blood stream (platelet compartment) being mimicked by different means.

In 2011, Balduini and her team reported a 3D system that represents the first spatial reconstruction of the bone marrow environment aiming at studying MK migration, adhesion to the sinusoidal vessel, proplatelets formation and platelets release (Pallotta et al.; “Three-Dimensional System for the In Vitro Study of Megakaryocytes and Functional Platelet Production Using Silk-Based Vascular Tubes”; Tissue Engineering: Part C Methods. 2011; 17(12): 1223-32). Silk microtubes (wall thickness of 50±20 μm to match proplatelet length, with pore sizes of 2-8 μm to allow proplatelet) were prepared with silk fibroin, a biologically derived protein polymer purified from domesticated silkworm (Bombyx mori) cocoons and then coated with SDF1-α (a chemoattractant) and Matrigel diluted with different proteins (von Willebrand factor (VWF), fibrinogen (FBG) or type I collagen). Suspension of 3·10⁵ MK, derived from cord blood (CB) hematopoietic stem cells (HSCs), was added at the interface between a collagen I gel preparation (mimicking the osteoblastic niche) and the outer wall of the coated silk tubes (mimicking the vascular niche). After a 16-hour incubation, 7%±2% of the seeded MK extended proplatelets through the microtube wall (with combination of VWF and FBG) and around 2 millions of platelets were collected in the solution perfused in the microtube at a 32 μL/mL flow rate (shear rate ˜40/s). In 2015, the 3D system was improved by embedding the silk tube (wall thickness of 50±20 μm and pore diameters of 22±4 μm) within a silk sponge (interconnected pores ranging from 100 to 500 μm in diameter) functionalized with extracellular matrix (ECM) proteins to fully recreate the physiology of human bone marrow niche environment (Di Buduo et al.; “Programmable 3D silk bone marrow niche for platelet generation ex vivo and modeling of megakaryopoiesis pathologies”; Blood. 2015; 125(14): 2254-2264). A total of 2.5·10⁵ mature CB-derived MK were seeded into the functionalized silk sponge. After 16 hours, the system allowed MK to migrate toward the vascular tube, adhere to the outer wall and after 24 hours proplatelets were extended into the lumen of the microtube. Platelets (1.4 million) were retrieved by perfusing a culture media in the microtube at a shear rate of 60/s. ECM components were used to functionalize the porous silk, such as type I collagen, fibrinogen, fibronectin, type IV collagen, or laminin, whereas microtubes were functionalized with fibronectin, type IV collagen and laminin. More recently, the same team (Di Buduo et al.; “Modular flow chamber for engineering bone marrow architecture and function”; Biomaterials. 2017; 146: 60-71) developed a simplified system consisting of a modular rectangular flow chamber, holding a silk sponge (3.4×20×5 mm; pore diameters 370±115 μm) functionalized with extracellular matrix components embedded in a central cavity connected to an inlet and an outlet to allow perfusion of the system. 3-4·10⁵ CB-derived mature MK were seeded for 24 hours within the functionalized silk scaffold. After this period, when culture medium was perfused at ˜90 μL/min (i.e., an average wall shear rate of 1.9 s⁻¹) for 8 hours, the flow allowed the detachment of platelet-like particles that were collected at the outlet. Even if the system was described as aiming at scaling up platelet production, the publication did not disclose any performance as regards this goal. This system was further used as a miniaturized bone tissue model for predicting drug response in patients, seeding stem cells (hematopoietic or induced pluripotent), i.e. precursor of MK, in the silk sponge (Di Buduo et al.; “Miniaturized 3D bone marrow tissue model to assess response to Thrombopoietin-receptor agonists in patients”; eLife 2021; 10:e58775). After 15 days or more, allowing stem cells to expand and differentiate into MK, few (4·10⁵) platelets were produced. Lastly, Tozzi et al. proposed to improve this simplified system by reintroducing functionalized channels (diameter 1 mm) in the silk sponge (pore size from 117±4.9 μm to 126±3.7 μm) (Tozzi et al.; “Multi-channel silk sponge mimicking bone marrow vascular niche for platelet production”; Biomaterials. 2018; 178: 122-133). Around 1.5·10⁶ CD41⁺CD42b⁺ CB derived MK were seeded within the silk scaffold drop-by-drop (190 MK/mm³ of silk) and cultured for 36 hours up to formation of proplatelets in the sponge. Then, a perfusion over 6 hours at a flow rate of 10 μL/min allowed the collection of 0.8-4.5 10⁶ of platelets in the lumen of the channels. The reason of adding the channels to the simplified system was to recreate the distinct environments of bone marrow i.e., to introduce different flow and shear stress between the sponge, the wall of the empty channels and the empty channels. When flow is introduced in the system, a homogeneous flow is observed within the channels whereas the flow is negligible within the scaffold and there is a sharp transition in shear values between the wall of channels and the silk sponge. Of note, the design of the bioreactor avoids “edge flows” between the scaffold and the bioreactor wall. All these silk devices integrate multiple sequential production steps—seeding of MK for end stage maturation (pseudopodial elongations) to form proplatelets, production and collection of platelets, rendering more difficult to optimize each step for scale up. For example, the seed density of MK cells in the microporous material is limited as MK need space to extend the proplatelets (190 MK/mm³ in Tozzi), thus requiring large volume of materials. In Tozzi, ˜1.3 m³ of silk sponge would be required to produce 3·10¹¹ platelets, the dose per patient per transfusion, thus requiring also large volume of media and products needed to functionalize the silk sponge. Stacking multiple devices does not solve the large amount of consumables needed and complicates the industrialization.

Avanzi et al. also described an integrated system to produce platelets from stem cells (WO 2012/129109 A2 (NEW YORK BLOOD CENTER, INC.) 27 Sep. 2012). The last step comprises a series of platelet release chambers. Each platelet release chamber is separated in an upper chamber that contains a 3D matrix or scaffold (with pores between about 2 μm and 6 μm, coated with factors that stimulate proplatelets formation and platelet release) and a lower chamber to collect the platelets. As in previous systems, MK are seeded on the scaffold. Here, two separate flows are applied in the upper and lower chambers, again to recreate the last steps of MK maturation into proplatelets within the vascular niche and platelet production in the blood stream. Proplatelet formation in the upper chamber and platelet collection in the lower chamber is conducted for about 1 to 2 days. Even if this system is promising in term of number of platelets collected per seeded MK, few MK (1·10⁵) can be seeded by chamber, releasing 1 to 3.3 10⁶ platelets (Avanzi M P et al.; “A novel bioreactor and culture method drives high yields of platelets from stem cells”; Transfusion. 2016; 56(1): 170-178).

Shepherd et al. in 2018 considered to construct structurally graduated collagen scaffolds that would provide a structural support for MKs but also have sieving capacity, based on differential cell size (high accessibility to the biggest cells in the top region whilst the base region was much less accessible to the biggest cells) (Shepherd J H et al.; “Structurally graduated collagen scaffolds applied to the ex vivo generation of platelets from human pluripotent stem cell-derived megakaryocytes: enhancing production and purity”; Biomaterials. 2018; 182: 135-144). The seed density of the MK cells was in the same range as previous systems (4·10⁵ MK loaded into the collagen scaffold in 5 mL of media) for overnight culture (19 hours+). The number of platelets produced per megakaryocyte that stayed in the bioreactor was 29.2±15, but lower when considering all MK introduced in the device. In addition, a concern about the quality of the produced platelets was expressed.

Microfluidic devices were also proposed, mimicking the porous structure of the endothelial cells of the bone marrow vasculature.

Nakagawa et al. disclosed two microfluidic bioreactors (Nakagawa et al.; “Two differential flows in a bioreactor promoted platelet generation from human pluripotent stem cell-derived megakaryocytes”; Experimental Hematology. 2013; 41(8): 742-748). In these systems, a flow in a specific direction applies a pressure on the megakaryocytes that are trapped in a porous structure. A second flow (main flow) is used to create the shear stress to release platelets at the outlet of the porous structure. The number of seeded MK were low (1.2·10⁵) and the number of platelets per MK seeded was lower than 1.

Thon et al. described a 2- then 3-channel bioreactor, with the walls between the medium channel and the upper or lower channels pierced with slits (0.1 to 20 μm) (WO 2014/107240 A1 (BRIGHAM & WOMENS HOSPITAL, INC.) Oct. 7, 2014; Thon et al.; “Platelet bioreactor-on-a-chip”; Blood. 2014; 124: 1857-1867; WO 2015/153451 A1 (BRIGHAM & WOMENS HOSPITAL, INC.) Aug. 10, 2015). MK introduced in the medium channel are pushed through the gaps where they are trapped and brought into contact with a flow in the upper and lower channels exposing them to a shear stress. In this system the range of shear rate was higher (100-2500/s) than with previous systems, with picks of shear rate occurring at gap junctions. Over 2 hours, the yield of PLT per megakaryocyte was 30 but the concentration of MK introduced in the system was low 1.9×10⁴±1.3×10⁴ MK per mL, due to the finite number of slits/gaps per device. To overcome the low capacity, the system was modified as disclosed in WO 2017/044149 A1 (BRIGHAM AND WOMEN'S HOSPITAL, INC.) 16 Mar. 2017) by replacing the slits/gaps between the “MK channel” (inlet channel) and the “platelet” channel (outlet channel) by a permeable membrane forming microfluidic pathways (pore sizes between 3 and 10 μm), tapering transversally at least one of the channels to allow decoupling pressure on MK and shear stress, as well as considering a plurality of inlet/outlet channels in parallel. Platelets are produced from the first hour and the number of platelets increases during the 24 hours of production. Then, possibility to recirculate fluids in each channel was added and the surface of the membrane has been increased either by lengthening the channels in S-shape or by adapting the membrane across a single reservoir vessel bioreactor recreating the two environments (MK reservoir and platelet reservoir) (WO 2018/165308 A1 (PLATELET BIOGENESIS, INC.) 13 Sep. 2018 ). The principle stays the same in all these improvements: the sizes of the membrane pores are smaller than the MK to prevent them to go through the membrane and the number of MK to be processed per device is limited by the number of pores available in the membrane. Thus, the system is scaled up by stacking multiple devices (WO 2020/018950 A1 (PLATELET BIOGENESIS, INC.) 23.01.202).

Lastly, in 2018, Eto and collaborators proposed a new culture system without the two compartments (US 2021/0130781 A1 (ETO et al.) May 6, 2021; Ito et al.; “Turbulence activates platelet biogenesis to enable clinical scale ex vivo production”; Cell. 2018; 174(3): 636-648). Based on in vivo observations performed on mouse bone marrow, they hypothesized that turbulent flow is a crucial physical factor for platelet release, in addition to the shear stress. They developed a liquid culture bioreactor which includes two mixing blades fixed at a horizontal angle to the power axis and at right angles to each other. The blades repeat an up-and-down reciprocal motion creating turbulence, shear stress and vorticity. Shear rate is below 60/s whatever the size of the bioreactor. Culture for maturation of MK derived from an immortalized cell line (imMKCL) generated from induced pluripotent cells (iPSC) was done with 1·10⁵ or 2·10⁵ imMKCL/mL in this bioreactor for 6 to 7 days in a basal medium with a cocktail of agents. With 8 L in the bioreactor, they achieved to have 100 billions of platelets. This method of liquid culture allows higher quantity of MK to be processed but is still mixing an MK maturation step and a production step, requiring a low initial concentration of MK.

Beyond bone marrow biomimetics and more in line with platelet production in the lung vasculature, other devices were developed.

Dunois-Lardé et al. showed that exposure of human mature MK to high shear rate on VWF surface led to cellular modifications resulting in platelets release within 20 minutes (Dunois-Lardé et al.; “Exposure of human megakaryocytes to high shear rates accelerates platelet production”; Blood. 2009; 27 Aug. 27; 114(9): 1875-83). Then, Blin et al. developed a rapid method (2 hours) for producing platelets in vitro from MK introduced in a microfluidic device which consists in a wide-array of VWF-coated micropillars distributed in parallel microchannels where a unique flow is applied (Blin et al.; “Microfluidic model of the platelet-generating organ: beyond bone marrow biomimetics”; Scientific Reports. 6, 21700 (2016)). At a high shear rate, free floating MK anchor on the pillars, elongate in proplatelets then releasing platelets. The suspension is recirculated in the device to give a new opportunity of anchoring to MK which have not met a pillar. The textured surface is defined by a 3D pattern on the channel wall as hexagonal array of disks in the plane and a 1D array of pillars (WO 2015/075030 A1 (PLATOD) 28 May 2015). To overcome higher density of anchored MK at entry of the channels which prevent to process high MK concentration because of the risk of clogging, larger space from pillar to pillar at the entrance of the channel and more narrow spaces while going further in the device was proposed (WO 2016/180918 A1 (PLATOD) 17 Nov. 2016). Surprisingly, in experiments using such micropillar multichannel device, increasing the height of the micropillars, aiming at offering additional surface for MK anchoring, did not result in a higher platelet yield, while it did not allow to increase the input MK concentration/volume.

Kumon et al. engineered curve-shaped 3D microchannels whose height gradually decreased along the flow pass to trap MKs of various sizes (Kumon et al.; “On-Chip Platelet Production Using Three Dimensional Microchannel”; 2018 IEEE Micro Electro Mechanical Systems (MEMS). 2018: 121-124). The trapped MK were exposed to fluid force in the microchannel and resulted in producing platelets. In such system, decreased height of the channel to 5 μm will rapidly lead the clogging of the device.

The one-flow microfluidics systems offer the possibility to synchronize platelet production in a very short period, but one issue is that the high shear rate to be applied in each channel require unrealistic large pumps when multiplying the number channels or stacking devices to achieve an industrial production.

When considering scaling up a platelet production at reasonable cost, the best system is a system that enables processing high volume of cells at high concentration, which in turn allows to use low quantity of culture medium and obtain high quantity of platelets. The platelet yield is another important parameter but varies for a same bioreactor (Ito et al.; “Turbulence activates platelet biogenesis to enable clinical scale ex vivo production”; Cell. 2018; 174(3): 636-648) according to many other factors (e.g. types of cells, clones, medium of culture, etc.).

### SUMMARY OF THE PRESENT INVENTION

The present invention relates to a method and platelet production device for large-scale platelet production from megakaryocytes. In particular, a device comprising a rotatable bed reactor containing a porous material has been used for producing platelets at large scale during a short time.

With the method according to the present invention, it is feasible to produce platelets during a short time of about 15 minutes. The platelet production may last for up to 12 hours, preferably for up to 4 hours.

Porous structures, as disclosed in European patent application EP 21155887, which are hereby incorporated by reference, comprise a macroporous material. The porous structures, therefore, function as obstacles for the flow and can be used as anchoring sites for the MK. The porous structures offered a scaffold for the MK to attach, while letting sufficient open spaces (the pores) for the MK to elongate when submitted to a shear stress. When the MK elongates, it then forms platelets. The porous structures may have a gradient in the pore density aiming at increasing the production of platelets. In this way, it is possible to prevent attachment of all the megakaryocytes only to the entrance of microstructures according to the flow direction and thus, to maximize the occupation of the porous structures by the megakaryocytes.

Accordingly, in embodiments of the present invention, the porous materials are defined as materials that contain pores (cavities, channels, interstices, etc.). The porous structures may be natural or artificial. The porous materials may be organic materials, inorganic materials, polymeric (plastic), metallic, ceramics and amorphous. The porous materials may comprise a combination of two or more materials. Porous materials may be made of one entity containing pores, or may be an assembly of several particles, beads, fibers or elements stacked together. The assembly of these particles forms a macroporous structure, in which the spaces in between the particles constitute the pores. The particles may be bonded, fused or glued to each other, or they may just be apposed in close proximity. The porous materials may have different additional nominations according to their structures: foams, fibres, bubble-like foamed materials, lattice or packed beads.

In embodiments of the present invention, the porous materials may have different pore sizes; also called pore width (diameter) which is the distance of two opposite walls of the pore; from 1 μm to 10 mm, preferably from 50 μm to 1 mm. A bulk of porous materials may have a same pore size or a range of pore sizes (i.e. different pore sizes) constituting a gradient. The bulk of porous material may be constituted from the same material or a combination of two or more materials with the same pore sizes or with different pore sizes.

In embodiments of the present invention, pores of the porous materials may be semi-close or open, preferably open and interconnected (through pores or connective pores) i.e. there are no dead-end or saccate (having the form of a sac or pouch). The pores may be of different shapes for example funnel shaped, cylindrical, roughness, ink-bottle-shaped or the like). The cross-sectional shape of a pore may be ovoid or polygonal (regular or not, smooth or straight, concave or convex). The pores of the porous material may be with ordered or irregular arrangement or a mixture of both. Porous materials may be prepared with different approaches.

In the embodiments of the present invention, the porous materials may be classified according to their porosity (ratio of the total pore volume Vp to the apparent volume V) as low porosity, middle porosity, or high porosity based on the number of pores per unit of volume. Generally, porous materials with low and middle porosity have closed pores. The porosity may span from 20% to 99.9%, preferably from 80% to 99.9%.

In embodiments of the present invention, the porous materials may be rigid or flexible. A more rigid structure, thus less deformed under the pressure of the flow, may act as a stronger anchor for the MK against the shear stress of the flow.

In embodiments of the present invention, the porous material may be coated with a ligand with affinity for megakaryocytes, e.g., (i) von Willebrand factor (VWF) or its functional variants, (ii) polypeptides comprising fragments of VWF, (iii) fibrinogen, (iv) fibronectin, (v) laminin, (vi) type IV collagen, (vii) type III collagen, (viii) type I collagen, and (ix) vitronectin.

In an embodiment, the porous material is coated by incubation with a solution of VWF or its functional variants. Typically, the concentration of VWF used for coating the solid phase is between 5 and 100 μg/mL. Preferably, the concentration of VWF is between 20 and 40 μg/mL. For example, the porous material may be coated with functional variants of VWF selected from the group consisting of recombinant wild-type VWF or mutated VWF polypeptides, expressed in E. coli or in mammalian cells, as monomeric or dimeric polypeptides.

In embodiments of the present invention, the platelet production device comprises a vessel containing a cell suspension, and a bed reactor containing a porous material, wherein the bed reactor is configured to be rotated while being immersed in the cell suspension. For example, the porous material comprises cotton fibers (100% organic cotton) that are cut into thin layers corresponding to the dimension of the cavity available in the bed reactor.

In embodiment of the present invention, the bed reactor comprises a hollow body including an outer peripheral wall extending from a base plate to a top plate such that a cavity is formed therebetween to accommodate the porous material.

The bed reactor further comprises a through hole that is disposed at the center of the bed reactor extending from the top plate to the base plate in order to facilitate a fluid flow through the bed reactor (e.g. the flow of the cell suspension). In this way, the cell suspension can enter the bed reactor via the through hole from both top and bottom plates of the bed reactor.

Preferably, the bed reactor has a symmetrical shape, e.g. a cylindrical shape.

The vessel is configured to be capped with a head plate (head cap) to thereby form a chamber. For example, the chamber is configured to be purged generating a controlled air composition.

The bed reactor is configured to be attached to a rotor shaft that is controlled by a rotor. For example, the rotor shaft is configured to pass through a central hole formed in the head cap.

Alternatively, the rotor shaft is configured to be coupled to the head cap through a shaft coupling mechanism.

The bed reactor is configured to be connected to the rotor shaft through a connector that is disposed on the top plate of the bed reactor. Apertures in the connector are configured to guide the flow of the cell suspension via the through hole into the bed reactor.

The cell suspension may directly be added into the vessel before placing the head cap. Alternatively, the cell suspension can be introduced through an opening in the head cap.

The head plate is configured to be secured on the vessel using e.g. by way of clamping the head plate onto the vessel.

Further, the cell suspension may be poured or pumped into the vessel using a peristaltic pump or other circulating pump.

The vessel is further configured to be jacketed using a cooling or heating jacket to control the temperature of the cell suspension therein.

The bed reactor comprises an outer wall that is made of a mesh. Alternatively, the outer wall comprises several openings.

The bed reactor may further comprise inner walls disposed within the hollow body and having a plurality of openings formed thereon.

The vessel may further comprise a baffle disposed therein to ensure circulation of the flow through the bed reactor.

The ratio between the volumes of the bed reactor and the vessel may be varied from just above 1:1 up to 1:100, preferably from 1:2 to 1:20.

For example, the bed reactor may contain up to 28 cm³ of porous material and is configured to fit in a 500 mL vessel. In particular, this ratio of the porous material to the volume of the vessel has been found to be a good match. Generally speaking, also higher amounts of porous material could be used. If so, also the vessel volume could be enlarged. Thus, other values instead of 28 cm³ of porous material and 500 mL for the vessel can be chosen.

The bed reactor is configured to be rotated for example at up to 1000 rpm.

The density of megakaryocytes per cubic millimeter of porous material may be in a range of 10·10³ MK/mm³ to 100·10⁶ MK/mm³, preferably in a range of 100·10³ MK/mm³ to 10·10⁶ MK/mm³.

In embodiments of the present invention, the method for producing platelets at large-scales using a platelet production device may comprise the steps of:
adding a cell suspension (i.e. a solution containing mature megakaryocytes) into a vessel of the platelet production device;
introducing a porous material into a bed reactor of the platelet production device;
mounting the bed reactor to a rotor shaft;
placing the bed reactor into the vessel of the platelet production device;
optionally closing the vessel with a head cap in order to maintain a controlled atmosphere;
rotating the bed reactor at a predetermined speed; and
optionally collecting samples of the cell and platelets suspension, for example at regular intervals, for counting and characterizing platelets and MK.

In embodiments of the present invention, the “cell suspension” for use in the method of the present invention for example may be obtained by the following steps:
providing stem cells selected from HSC (e.g., from umbilical cord, peripheral blood or bone marrow), engineered HSCor from the group consisting of embryonic stem cells, engineered embryonic stem cells, induced pluripotent stem cells, and engineered induced pluripotent stem cells;
culturing the stem cells, i.e. expanding the cells and differentiating the expanded cells into MK.

In embodiments of the present invention, the step of introducing the porous material into the bed reactor comprises the step of filling the whole volume of the bed reactor in bulk. Alternatively, the porous material is arranged in an assembly of several layers of a few hundred micrometers, wherein the porous material may be separated by intermediate walls disposed within the bed reactor, e.g. in parallel to the top and base plates. For example, in cases where the volume of the vessel is not filled with the layered porous material, the bed reactor may be filled with inserts, e.g. plastic inserts, thereby establishing the flow only through the layers porous material.

### BRIEF DESCRIPTION OF THE DRAWINGS

The present invention will now be described by way of example with reference to the accompanying drawings.

It is shown in

FIGS. 1A, 1B: Schematic representation of a large-scale platelet production device with two different configurations: 1A) a rotor shaft that passes through a hole in a head cap is connected to a bed reactor; 1B) a rotor shaft coupled to the head cap via a shaft coupling mechanism is connected to a bed reactor.

FIGS. 2A-2C: 2A) A 3D representation of the bed reactor. 2B-C) A cross-view representation of the bed reactor filled with the porous material. Arrows show the direction of the flow through a through hole at the center of the bed reactor. 2B) and 2C, respectively, representing the bed reactor that is filled with a bulk porous material and a layered porous material.

FIGS. 3A, 3B: Schematic representation of the platelet production device that is coupled to a downstream sorting device. 3A) The downstream sorting device is connected through a tubing and a pump. 3B) The downstream sorting device is connected directly to the vessel of the platelet production device.

FIG. 4: Schematic representation of a small-scale platelet production device. A cavity of 6.6 mm×35.5 mm×0.1 mm micromachined in Poly(methyl methacrylate) (PMMA).

FIGS. 5A-5C: Schematic representation of the arrangement of the porous material in the bed reactor as used for the experiments. 5A) A lateral cross-sectional view of the bed reactor with only two thin layers of the porous material (e.g. cotton) placed mid-height of the bed reactor; 5B) A lateral cross-sectional view of the bed reactor without the porous material; 5C) A top cross-sectional view of the bed reactor filled with two layers of the porous material symmetrically placed in the bed reactor.

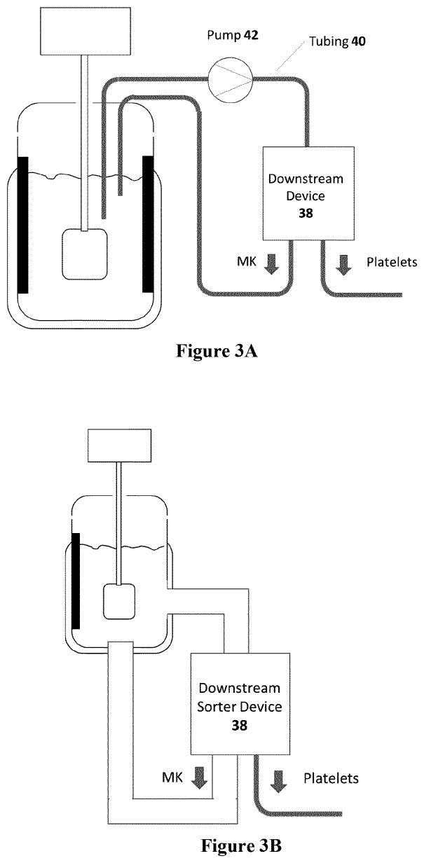

FIG. 3: Experimental results indicating that the platelet numbers increase from the start of the production experiment: Influence of the rotation speed of the bed reactor in a large-scale setup with porous material and a comparison with a small-scale setup including porous material (mean+/−SEM; n=2).

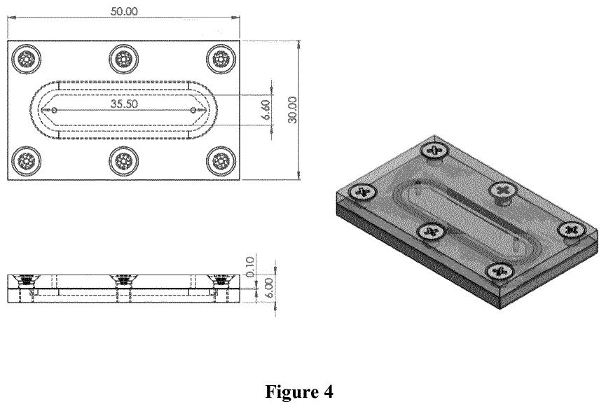

FIG. 4: Experimental results indicating that the platelet yield (the highest number of platelets over MK CD41/CD42+ number at the start of the platelet production) increases in a large-scale setup with porous material compared to a small scale-setup with porous material (mean+/−SEM; n=2).

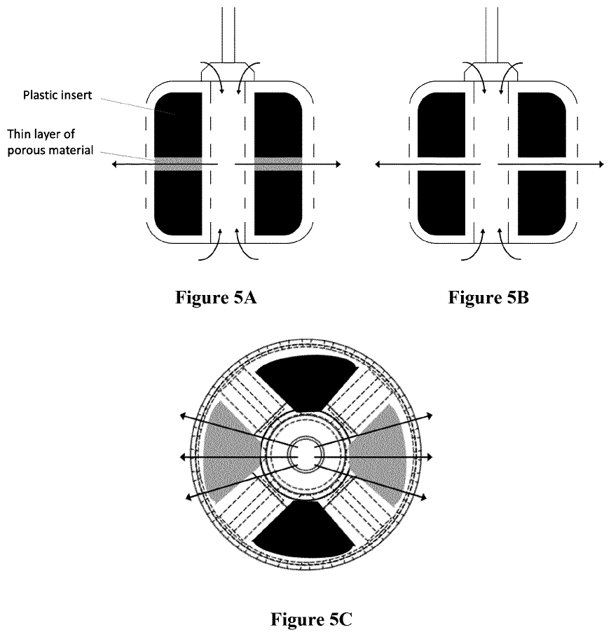

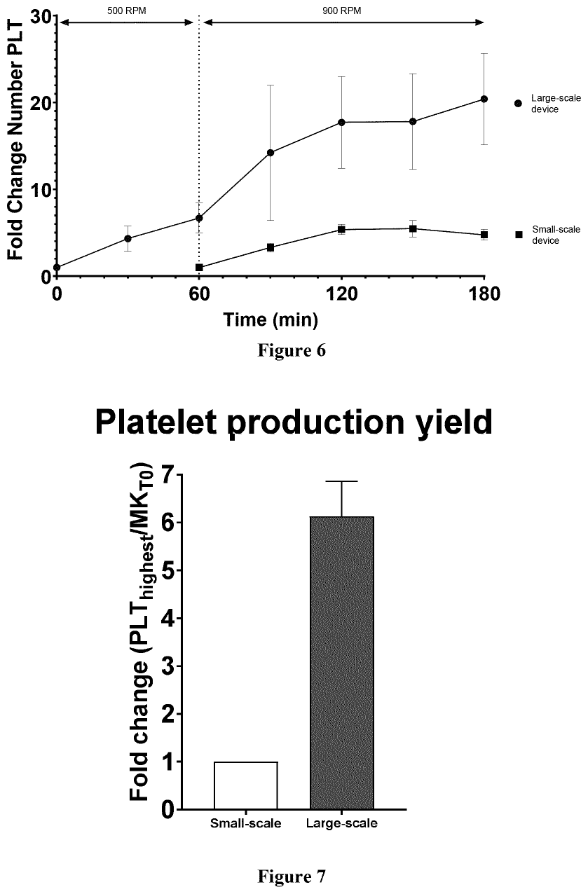

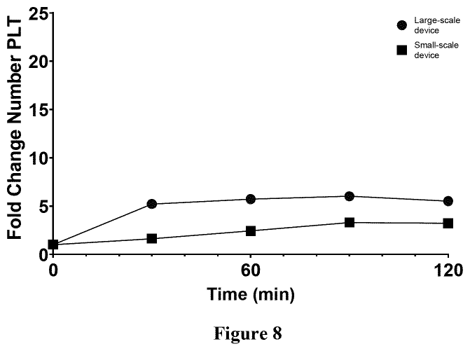

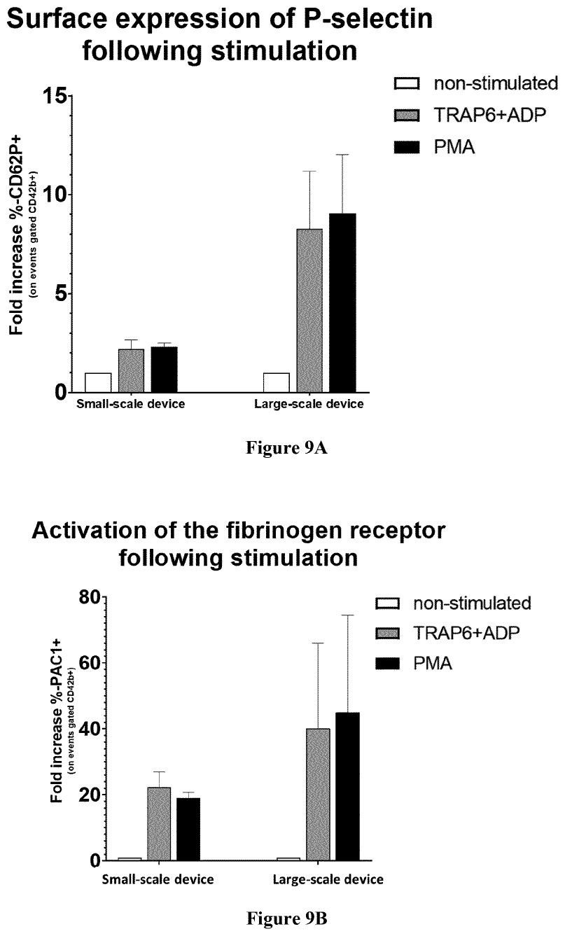

FIG. 5: Experimental results showing a comparison of the platelet numbers in a small-scale setup with porous material and a large-scale setup without porous material at a rotation speed of 900 rpm.

FIGS. 6A, 9B: Characterization of platelets upon activation. Comparison of the platelets at end of the production in the small-scale production and large-scale production setups both with porous material (mean+/−SEM; n=2).

### DETAILED DESCRIPTION

To produce platelets, megakaryocytes (MK) are initially derived from hematopoietic progenitors restricted to the megakaryocytic lineage.

The primary signal for megakaryocyte production is thrombopoietin (TPO), TPO receptor agonist or TPO mimetic peptides. TPO is necessary for inducing differentiation of progenitor cells in the bone marrow towards a final megakaryocyte phenotype. Other molecular signals for megakaryocyte differentiation include for example GM-CSF, IL-3, IL-6, IL-11, Flt-3 ligand, SCF.

MK progenitor cells can be obtained by in vitro culture.

As used herein, the term “cell suspension” denotes a solution containing mature MK ready to produce platelets obtained by in vitro culture. This cell suspension may also contain MK progenitors, proplatelets and platelets.

Preferably, said “cell suspension” for use in the method of the present invention is obtained by the following steps:
a) providing stem cells for example selected from HSC (e.g., from umbilical cord, peripheral blood or bone marrow), engineered HSC or from stem cells selected from the group consisting of embryonic stem cells, engineered embryonic stem cells, induced pluripotent stem cells, and engineered induced pluripotent stem cells;
b) culturing said stem cells, i.e. expanding the cells and differentiating the expanded cells into MK.

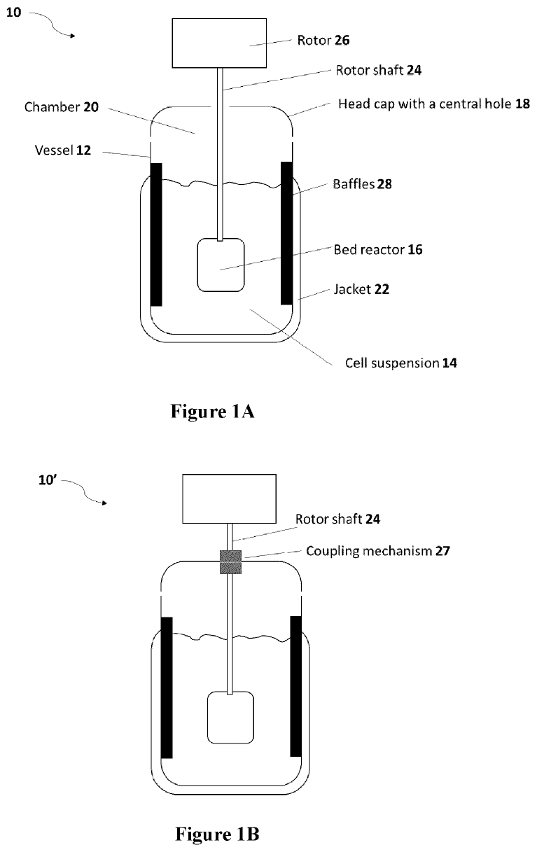

FIGS. 1A and 1B indicate a platelet production device 10, 10′ (i.e. a reactor) for large-scale production of platelets.

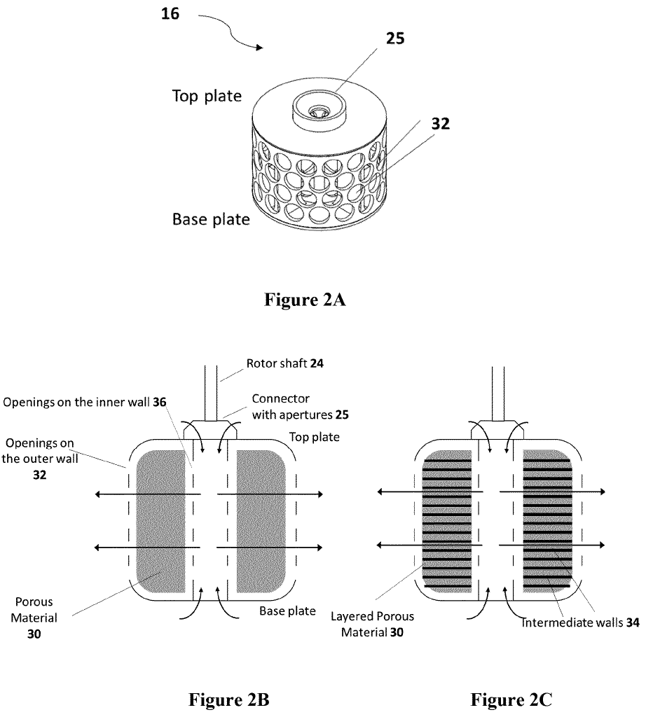

The platelet production device 10, 10′ comprises a vessel 12 configured to contain the cell suspension 14 and a bed reactor 16 configured to accommodate a porous material 30 as shown in FIGS. 2B and 2C.

The bed reactor 16 is in the mounted state located in the vessel 12.

The vessel 12 can be capped with a head plate or head cap 18 to thereby form a chamber 20. This way, the air composition in the chamber can be controlled. For example, the air composition is composed of 5% CO₂.

To control the temperature of the cell suspension 14, the vessel 12 can be jacketed using a cooling or heating jacket 22. Alternatively, any other temperature suitable control units can be used for controlling the temperature.

For example, a jacketed vessel was used with circulating water thermo-regulated at 37° C.

The cell suspension 14 is directly added into the vessel 12 before placing the head cap 18. Alternatively, the cell suspension can be introduced through an opening in the head cap 18.

Further, the cell suspension 14 can be poured or pumped into the vessel 12 using a peristaltic pump or other circulating pump.

At the end of the experiment, the cell suspension 14 now also containing produced platelets can be drained out of the vessel 12 using a drain output at the bottom of the vessel (not shown).

Alternatively, the cell suspension 14 including the platelets can be pumped out of the vessel through a tubing plunged into the vessel 12.

The bed reactor 16 is configured to be immersed into the cell suspension 14.

The bed reactor 16 is configured to be attached to a rotor shaft 24 controlled by a rotor 26. In this way, the bed reactor 16 can be rotated at high speeds.

The rotor shaft 24 may pass through a central hole in the head cap 18 as indicated in FIG. 1A. Alternatively, the rotor shaft 24 may be coupled to the head cap 18 through a shaft coupling mechanism 27. In this case, the head cap does not have the central hole. For example, the head cap can be secured to the vessel via a clamping process.

FIG. 2A indicates a 3D representation of the bed reactor 16. FIGS. 2B and 2C indicate cross-sectional views of the bed reactor 16, respectively, filled with a bulk porous material 30 and a layered porous material 30. Arrows show the direction of the flow.

The bed reactor 16 comprises a hollow body including an outer peripheral wall extending from a base plate to a top plate such that a cavity is formed therebetween to accommodate the porous material.

The bed reactor 16 further comprises a through hole that is disposed at the center of the bed reactor and extends from the top plate to the base plate in order to facilitate a flow therethrough as indicated by the arrows in the FIGS. 2B and 2C.

The bed reactor 16 is configured to be connected to the rotor shaft 24 through a connector 25 disposed at the top plate. Apertures in the connector 25 allow the flow to pass through the connector 25 into the through hole at the center of the bed reactor 16.

FIG. 2A indicates that the peripheral outer wall of the bed reactor 16 is made of a mesh. Alternatively, the outer wall may comprise several openings 32. The provision of the openings 32 allows a better contact between the cell suspension 14 and the porous material 30 contained in the bed reactor 16.

The bed reactor 16 may further comprise inner walls disposed within the hollow body and having a plurality of openings 36 formed thereon as indicated in FIGS. 2B and 2C. This may further enhance the contact between the porous material and the cell suspension by allowing the flow to circulate through the bed reactor 16 while the bed reactor being rotated. In this way, the rotating of the bed reactor induces a radial flow through the bed reactor.

The provision of the central through hole in the bed reactor (to thereby allow the flow to centrally enter from the top and bottom sides of the bed reactor) together with the openings 32, 36 on the inner walls and the peripheral outer wall ensure a path for the flow, which enters at the center of the bed reactor and radially exits the bed reactor while passing through the porous material. This in turn, results in an optimum repartition of the flow through all the height of the bed reactor 16. This effect is further improved in the bed reactors of the cylindrical shape.

In order to keep the chamber 20 sterile, the rotor shaft 24 can also be connected to the bed reactor 16 through a magnetic shaft coupling.

Baffles 28 are provided in the vessel 12 to prevent swirling and vortexing when rotating the bed reactor 16 at high speeds. This ensures circulation of the flow through the bed reactor 16.

When the cell suspension 14 flows radially through the porous material 30, the MK can attach to the microstructure of the porous material.

The MK caught into the porous material will be submitted to shear stress and/or a pulling force. Under shear stress, the cytoplasm of the MK elongates and forms platelets. The higher the rotational speed, the higher is the flow velocity through the porous material, resulting in a higher shear stress.

The flow velocity through the porous material 30 is also proportional to the hydraulic resistance of the porous material.

As the platelets are produced, they are released in the cell suspension 14, and are carried by the flow, circulating freely into the vessel 12.

The MK attach to the porous material by cell surface binding to the porous material or by its coating (e.g. thanks to integrins at its surface), or by getting caught into the interstices of the porous material.

The MK attachment may or may not be permanent. The MK may detach from the porous material and may get reattached when recirculating through the porous material.

The porous material 30 may fill the whole volume of the bed reactor 16 in bulk as shown in FIG. 2B. Alternatively, it may be arranged in an assembly of several layers of a few hundred micrometers, separated or not by intermediate walls 34 as shown in FIG. 2C.

The presence of intermediate walls 34, while reducing the available space for the porous material 30 in the bed reactor 16, results in additional wall shear rate.

The bed reactor 16 can be scaled up to increase platelet production. The vessel 12 would be scaled up accordingly to fit the large bed reactor 16. Preferably, the bed reactor can have a volume from 1 mm³ up to 1 m³. The vessel 12 must be large enough to fit the bed reactor, but 30 it can be larger as well.

The ratio between the volumes of the bed reactor and the vessel can be from just above 1:1 up to 1:100, preferably 1:2 to 1:20.

For example, the bed reactor 16 can contain up to 28 cm³ of porous material 30 and can fit in a 500 mL vessel 12.

The larger the bed reactor, the higher is the inertia, so the lower is the maximum rotational speed it can reach. Preferably, the 28 cm³ bed reactor can be rotated at up to 1000 rpm.

To optimize the platelet production yield per MK, the MK density per cubic millimeter of porous material needs to be in a range of 10·10³ MK/mm³ to 100·10⁶ MK/mm³, preferably in a range of 100·10³ MK/mm³ to 10·10⁶ MK/mm³.

If the volume ratio between the bed reactor and the vessel is just above 1:1, the cell suspension may contain 10·10³ MK/mL up to 100·10⁶ MK/mL. Alternatively, if the volume ratio between the bed reactor and the vessel is 1:100, the cell suspension may contain 0.1·10³ MK/mL up to 1·10⁶ MK/mL.

Thanks to the high-speed rotation of the bed reactor, the cell suspension recirculates at a high rate through the porous material, maximizing the chances for MK to attach to the porous material and form platelets. This way, the platelet production can be performed in a limited amount of time.

From the moment where the cell suspension is introduced into the vessel and the bed reactor starts rotating, the platelets are produced in as short as 15 minutes and production can last for up to 12 hours, preferably for 4 hours.

The platelet production increases over time. This can be monitored by sampling the cell suspension and performing a platelet count with flow cytometry or any cell counter. Overtime, the platelet count increases, and the MK count decreases.

The platelets can be produced in batch process or continuous process. Indeed, as the platelet production progresses, the MK count decreases, and more MK can be added to the vessel.

The porous materials may be organic materials, inorganic materials, polymeric (plastic), metallic, ceramics and amorphous. They may be composed from a combination of two or more materials. The porous materials may be having different additional nominations according to their structures: foams, fibres, bubble-like foamed materials, lattice or packed beads.

The porous materials may have different pore sizes from 1 μm to 10 mm, preferably from 50 μm to 1 mm.

A bulk of porous materials may have the same pore sizes or a range of pores sizes constituting a gradient. A bulk may be constituted from the same material or a combination of two or more materials with the same pore sizes or with different pore sizes.

For example, the porous material is made of 100% cotton fibers.

The porous material may be coated with a ligand with affinity for megakaryocytes, e.g., (i) von Willebrand factor (VWF) or its functional variants, (ii) polypeptides comprising fragments of VWF, (iii) fibrinogen, (iv) fibronectin, (v) laminin, (vi) type IV collagen, (vii) type III collagen, (viii) type I collagen, and (viii) vitronectin.

The porous material may be coated by incubation with a solution of VWF or its functional variants. Typically, the concentration of VWF used for coating the solid phase is between 5 and 100 μg/mL. Preferably, the concentration of VWF is between 20 and 40 μg/mL.

Further, the porous material may be coated with functional variants of VWF selected from the group consisting of recombinant wild-type VWF or mutated VWF polypeptides, expressed in E. coli or in mammalian cells, as monomeric or dimeric polypeptides.

Advantageously, the rotating bed reactor allows to work under sterile conditions. For example, said reactor is sterile.

The method for producing the porous material according to the present invention may comprise the step of sterilization of the porous material. This sterilization step may occur before or after the sealing of the reactor chamber.

The platelet production device according to the present invention may further comprise optional means such as:
(a) an upstream sorter and/or a mixer for enriching the suspension with megakaryocytes and homogenizing cell concentration of said suspension of megakaryocytes, upstream of the platelet production device,
(b) optionally a sorter downstream from the platelet production device for purifying the outflow suspension by sorting platelets from megakaryocytes or other cell residues,
(c) optionally a platelet concentrator downstream from the platelet production device for concentrating the outflow suspension

The use of the upstream sorter (i.e. a megakaryocyte sorter) may be advantageous to obtain a suspension homogeneous in terms of MK population and to obtain consistent yield and quality for industrial production of platelets. In particular, the upstream sorter includes means to separate the MK from platelets and other cell residues. A conventional cell sorter as described below can be used as the downstream sorter (e.g. a platelet cell sorter) allowing to obtain a cell suspension enriched in MK in a pre-defined proportion, from 10% to about 100%.

At the outlet of the platelet production device, the outflow contains produced platelets, but it may further contain naked nuclei and/or intact MK. A means for separating the produced platelets may be thus advantageously put downstream of the platelet production device. A conventional cell sorter device may be used, such as an elutriation rotor or a leukoreduction filter used in apheresis techniques. The downstream sorter may also be a filtration device used for large-scale filtration in industry such as a membrane filtration, a tangential flow filtration device, such as hollow fibers or a spinning filter.

The step of separation can occur at the end of platelet production, or can be performed at different intervals during the production, or in a continuous mode during platelet production. Separating platelets from MK during platelet production, allows to extract the produced platelets as they are formed, thus preventing them from staying for too long in the platelets production device, which may affect their quality.

In a batch process mode, the downstream sorter may be independent from the platelet production device. At the end of the platelet production, the cell and platelet suspension is transferred using a pump to the cell sorter device.

In a semi-continuous or a continuous process, the downstream sorter device may be connected through a tubing or a pipe to the vessel of the platelet production device.

FIG. 3A illustrates a downstream sorter device 38 which is connected through a tubing 40 and a pump 42 to the vessel 12. In this case, the cell and platelet suspension circulate through the downstream sorter device 38 and the MK isolated in the cell sorter device are reintroduced into the vessel 12.

FIG. 3B illustrates another configuration that the downstream sorter device 38 is connected directly to the platelet production device 10, 10′, without the use of a pump. In this configuration, the rotation of the bed reactor induces a displacement of the flow that is used to transfer the cell and platelet suspension to the cell sorter device.

At the outlet of the platelet production device or at the outlet of the sorter, the outflow suspension may be concentrated to reach platelet concentration suitable for human injection. Conventional cell concentrator device may be used, such as hemodialysis or tangential flow filtration device. The platelets are washed, to remove the cell culture medium and the platelets are re-suspended into a storage solution, such as Platelet Additive Solutions (PAS) as PAS-A, PAS-B, PAS-C, PAS-D, PAS-E or PAS-G (Ashford et al.; “Standard terminology for platelet additive solutions”; The International Journal of Transfusion Medicine; Vol. 98, Issue 4, (2010). p. 577-578), with or without an addition of human plasma.

The present invention will be further understood in light of the following non-limiting examples, which are given for illustration purposes only, and also in connection with the attached drawings.

### Examples

Material and Methods

CD34+ Cells Culture and Differentiation

CD34+ cells were isolated human cord blood (CB) by an immunomagnetic technique (Miltenyi Biotec, Paris, France) as previously reported (Poirault-Chassac et al., “Notch/Delta4 signaling inhibits human megakaryocytic terminal differentiation”; 2010; Blood December 16; 116(25):5670). CD34+ cells were cultured in 6-well plates (Sarstedt, 83.3920.500), in a humid atmosphere at 37° C. in 5% CO₂ in complete medium consisting of Iscove modified Dulbecco medium (IMDM; Gibco Life Technologies, 31980022) supplemented with 15% BIT 9500 serum substitute (Stem Cells Technologies, 09500), α-monothioglycerol (Sigma-Aldrich, M6145-25ML) and liposomes (3L-a-Phosphatidylcholin Dipalmytyol (P0763-250MG), cholesterol (C3045-5G) and oleic acid (O3880-1G); Sigma Aldrich and Bovine Serum Albumine (BSA) Fraction V from PanReac (A2244.0050)). Human recombinant stem cell factor (SCF)(6.25 U/mL; Miltenyi Biotec, 130-096-696), Interleukin-3 (IL-3) (10 U/mL; Miltenyi Biotec, 130-095-068) and thrombopoietin peptide agonist (TPO) (10 nM; synthetized by Sigma Aldrich) were added once at day 0 to the culture medium. At day 6, cells are centrifuged and resuspended in fresh complete medium supplemented with 50 nM TPO and 0.5 U/mL SCF for 5 to 7 additional days.

Small-Scale Setup for Platelet Production

Small-Scale Production Device

The small-scale platelet production device served as a control in all examples (FIG. 4). It is a cavity of dimension 6.6 mm×35.5 mm×0.1 mm micromachined in Poly(methyl methacrylate) (PMMA). The cavity is closed with a lid made of PMMA. The cavity is filled with porous material, it has one inlet and one outlet so it can be connected to the peristaltic pump used to circulate the cell suspension through the porous material.

Small-Scale Production System Architecture

The cell suspension was placed into a 50 mL Falcon tube fixed on an orbital mixer (IKA MS3 basic), rotating at least at 300 rpm. A peristaltic pump (IPC8, ISMATEC, Germany) was used to flow the cell suspension through the platelet production device. Both inlet and outlet tubings arrived in the same container containing the cell suspension, leading to MK recirculation. The said container was connected to the inlet and outlet of the small-scale device with flexible 0.57 mm ID tubing (Tygon ST R-3607, Idex Health and Science, Germany). The cell suspension circulated through the small-scale device at a rate of 0.94 mL/min for 2 hours. The whole setup for small-scale platelet production was enclosed into a chamber, thermo-regulated at 37° C. by an air controller (The Box, Life Imaging Services, Switzerland). Samples of the cell and platelets suspension were collected from the Falcon tube with a micropipette at regular intervals during the platelet production process for platelets and MK count and characterization.

Large-Scale Setup for Platelet Production

The large-scale platelet production device was a jacketed 500 mL-glass baffled vessel (Vessel V2, SpinChem, Sweden), in which a 28 cm³ bed reactor (RBR S2, Spinchem, Sweden) was placed and attached to the rotor shaft (rotor IKA RW 20 digital). The rotor can reach a rotational speed of up to 1000 rpm. The head cap, with a central aperture through which the rotor shaft passes, was clamped on the vessel. A gas mixer unit (CO₂ Biobrick, Life Imaging Services, Switzerland) provided air with 5% CO₂ at a rate of 22 L/hour in the chamber. The jacket around the vessel was connected to a temperature control unit (Cobra) set at 37° C., which circulated thermo-regulated water through the jacket.

Filling this whole 28 cm³ bed reactor with the porous material would allow to get 28·10¹¹ MK at a concentration as high as 100·10⁶ MK/mm³ in the porous material during production. In this case, the suspension of MK would be at a concentration as high as 5.6·10 9 MK/mL in the 500 mL vessel.

The bed reactor can be customized to contain thin layers of the porous material in order to study the impact of the amount of the porous material on the platelet production.

In two experiments, two thin layers of the porous material having dimensions of 179.61 mm²×0.2 mm were placed at mid height of the bed reactor (FIG. 5A). The rest of the bed reactor was filled with plastic inserts, so that the flow only flows through the thin layers of porous material. The thin layers of the porous material were shaped as quadrants and were symmetrically positioned within the bed reactor, as seen in the vertical cross-view of the bed reactor (FIG. 5C). The total amount of porous material was therefore 3.2 times higher than in the small-scale platelet production device.

In one another experiment, the above bed reactor was used without porous material (FIG. 5B). Samples of the cell and platelets suspension were collected at regular intervals during the platelet production process for platelets and MK counts and characterizations. The rotor was interrupted only briefly during the sample collection, which was performed using a pipette.

Preparation of the Porous Materials

Cotton fibers (100% organic cotton) were cut into thin layers at the dimension of the cavities of the small-scale and large-scale devices respectively.

Protein Surface Treatment

The Human von Willebrand factor (VWF) used for coating the porous material was Wilfactin (LFB, Les Ulis, France). It was diluted at 40 μg/mL in phosphate buffered saline (PBS 1×) without calcium and magnesium ions and perfused in the small-scale devices or poured on the pieces of porous material intended for use in the large-scale reactor. The small-scale devices and the pieces of porous material were incubated overnight at 4° C. in a humid chamber.

Preparation of the Cell Suspensions

After 11, 12 or 13 days of culture, the cells were collected from the 6-well plates and transferred to a 50 mL tube. The cell concentration was estimated by a manual count (using a Malassez cell counting chamber). From this initial cell suspension, a volume of 5 mL was collected and transferred into a 50 mL tube for use in the small-scale production setup. Because the amount of cotton fibers was 3.2 times higher in the large-scale setup compared to the small-scale setup, we targeted an absolute cell number about 3.2 times higher in the cell suspension used for the large-scale setup. The cell suspension was therefore adjusted accordingly and complemented with IMDM (Gibco Life Technologies, 31980022) to reach a volume of 200 mL for use in the large-scale platelet production device.

Methods for the Characterization of Collected Platelets

Collected platelets were characterized using a flow cytometer BD Fluorescence Accuri C6 PLUS (BD Biosciences, Le Pont de Claix, France). Surface specific antigens of platelets were analyzed with fluorescein isothiocyanate (FITC)-conjugated anti-human CD41 (αIIb) and R-phycoerythrin (PE)-conjugated anti-human CD42b (GPIba) (both from Beckman Coulter, Villepinte, France). Platelets were incubated for 20 min in the dark at room temperature (RT) with the fluorescence-conjugated monoclonal antibodies. Controls were performed using FITC mouse IgG₁ (Beckman Coulter), PE mouse IgG₁ (BioLegend, San Diego, CA, USA). Platelets were defined as acquired events being (i) smaller than 7 μm (gated based on forward scatter properties and calibrated beads from Spherotech, Libertyville, IL, USA) and (ii) double positive to CD41 and CD42b labelling (CD41⁺/CD42b⁺).

Activation of the collected platelets was assessed with FITC-conjugated anti-human activated αIIbβ3 (PAC1 clone) (BD Biosciences) and allophycocyanin (APC) anti-human CD62P (BD Biosciences) with platelet collected at the end of production (120 min for the small-scale setup and 180 min for the large-scale setup). Platelets were activated in home-made Tyrode's buffer (140 mM NaCl, 1 mM MgCl₂, 10 mM HEPES, 1 mg/mL bovine serum albumin, 5.5 mM glucose, 2 mM CaCl₂ pH adjusted to 7.4 with NaOH) and incubated for 30 min in the dark at RT with PAC1-FITC, CD62P-APC, and CD42b-PE. Activation was performed with either (i) 40 μM Thrombin Receptor Activator Peptide-6 ([TRAP-6], Bachem, Bubendorf, Switzerland) plus 100 μM adenosine diphosphate ([ADP], Merck Sigma Aldrich, St. Louis, MO, USA), or (ii) Phorbol 12-myristate 13-acetate ([PMA], Merck Sigma Aldrich). Controls were performed with Arg-Gly-Asp-Ser ([RGDS], Merck Sigma Aldrich), PE mouse IgG₁ (BioLegend) and APC mouse IgG1 (Biolegend).

Results

Two experiments were run successively on the same day for studying the effect of the rotational speed in the large-scale setup and also comparing the large-scale setup to the small-scale setup, both with porous material. In the first experiment, the total number of MK CD41/CD42+ in suspension introduced in the large-scale setup was 8.10·10⁶ versus 9.89·10⁵ in the small-scale and, respectively 9.82·10⁶ versus 1.15·10⁶ in the second experiment. In the large-scale setup, the bed reactor was first rotating at 500 rpm for 60 minutes, followed by 2 hours at 900 rpm. The increase in rotational speed induces a higher flow velocity through the porous material. Increasing the rotational speed of the bed reactor resulted in an increase in platelet production (FIG. 6): Over 60 minutes, the average increase in the platelet production was 1.94 times higher at 900 rpm compared to 500 rpm. During the 2 hours of the experiment at 900 rpm in the large-scale setup, the number of platelets increased continuously, whereas the maximum of production was reached between 60 and 90 min in the small-scale setup and then plateaued. When considering the highest number of the platelets produced by each setup, the yield of production, i.e. the number of platelets produced over the number of MK CD41/CD42+ introduced at the start of the experiment was up to 6-fold higher with the large-scale setup than with the small-scale setup (FIG. 7).

The third experiment compared the platelet production in the small-scale device with porous material and the large-scale device that did not contain any porous material. In this way it was possible to estimate indirectly, using the same small-scale control, whether there is a benefit of adding the porous material in the bed reactor. The total number of MK CD41/CD42+ in suspension introduced in the large-scale setup was 10.78·10⁶ i.e., in the same range of the numbers in the previous experiments, versus 3.41·10⁶ in the small scale. In the large-scale device, the bed reactor was rotating at 900 rpm. Over a period of 120 minutes, the platelet production was slightly higher in the large-scale setup compared to the small-scale device (FIG. 8), but the difference between the two setups was much smaller than that observed in the previous experiments where porous material was added in the large-scale setup.

At the end of the two first experiments (120 min for the small-scale setup and 180 min for the large-scale setup), the collected platelets were stimulated either with TRAP6+ADP or with PMA. Activation endpoints such as surface expression of P-selectin (FIG. 9A) and activation of the fibrinogen receptor αIIbβ3 with PAC1 binding (FIG. 9B) were monitored. The platelets from both setups have responded to stimulation by increasing their expression of P-selectin (positivity to CD62P) and activation of the fibrinogen receptor (positivity to PAC1), compared to baseline (non-stimulated). In addition, for both activation markers, a better activability of the platelets produced in the large-scale setup with the porous material was observed compared to the small-scale setup, highlighting the better quality of the produced platelets in the large-scale setup.

1. A platelet production device for large-scale platelet production from megakaryocytes, comprising:
a vessel and a rotatable bed reactor disposed therein,
wherein the bed reactor is configured to contain a porous material and to be immersed into a suspension cell contained within the vessel.

2. The device of claim 1, wherein the cell suspension comprises a solution including mature megakaryocytes, and wherein the porous material comprises foams, fibers, 3D printed porous structures, woven filters, non-woven filters, microcarriers, gels or hydrogels or packed-beads.

3. The device of claim 1, wherein the bed reactor comprises a through hole disposed at the center of the bed reactor for facilitating a flow of the cell suspension through the bed reactor.

4. The device of claim 1, wherein the bed reactor comprises a hollow body including a peripheral outer wall extending from a base plate to a top plate, and wherein the peripheral outer wall comprises openings or is made of a mesh, preferably the hollow body has a cylindrical shape.

5. The device of claim 4, wherein the bed reactor further comprises inner walls disposed within the hollow body and extending from the base plate to the top plate, wherein the inner walls each include a plurality of openings formed thereon.

6. The device of claim 1, wherein the vessel is configured to be capped with a head cap forming a chamber, preferably the chamber is configured to be purged.

7. The device of claim 1, wherein the bed reactor is configured to be attached to a rotor shaft via a connector, wherein the connector comprises apertures for facilitating the cell suspension to enter into the bed reactor.

8. The device of claim 1, wherein the head cap comprises an opening for adding the cell suspension into the vessel.

9. The device of claim 8, wherein the device further comprises a pump for pumping the cell suspension into the vessel, preferably the pump includes a peristaltic pump.

10. The device of claim 1, wherein the vessel is further configured to be jacketed using a cooling or heating jacket.

11. The device of claim 1, where the bed reactor is configured to be rotated at up to 1000 rpm.

12. The device of claim 1, wherein the bed reactor further comprises intermediate walls configured to be disposed between layers of the porous material arranged in the bed reactor.

13. The device of claim 1, wherein the vessel comprises a baffle disposed therein.

14. The device of claim 1, wherein the ratio between the volumes of the bed reactor and the vessel varies from 1:1 up to 1:100, preferably from 1:2 to 1:20.

15. The device of claim 1, wherein the bed reactor comprises up to 28 cm³ of porous material and preferably, is configured to fit in a 500 mL vessel.

16. The device of claim 1, wherein the density of megakaryocytes per cubic millimeter of porous material is in a range of 10·10³ MK/mm³ to 100·10⁶ MK/mm³, preferably in a range of 100·10³ MK/mm³ to 10·10⁶ MK/mm³.

17. The device of claim 1, wherein the porous material is coated with a ligand having affinity for megakaryocytes, preferably the ligand comprises von Willebrand factor or its functional variants, polypeptides comprising fragments of Willebrand factor, fibrinogen, fibronectin, laminin, type IV collagen, type III collagen, type I collagen, or vitronectin.

18. A method for producing platelets at large-scales using a platelet production device according to claim 1, comprising:
adding a cell suspension into a vessel of the platelet production device;
introducing a porous material into the bed reactor of the platelet production device;
mounting the bed reactor to a rotor shaft;
placing the bed reactor into the vessel of the platelet production device;
optionally closing the vessel with a head cap in order to maintain a controlled atmosphere;
rotating the bed reactor at a predetermined speed; and
optionally collecting samples of the cell and platelets suspension for counting and characterizing the platelets and MK.

19. The method of claim 18, wherein the method further comprises obtaining the cell suspension by the following steps:
providing stem cells selected from HSC, engineered HSC or from the group consisting of embryonic stem cells, engineered embryonic stem cells, induced pluripotent stem cells, and engineered induced pluripotent stem cells; and
culturing the stem cells for expanding the cells and differentiating the expanded cells into MK.

20. The method of claim 18, wherein the step of introducing the porous material into the bed reactor comprises the step of filling the whole volume of the bed reactor with a bulk of the porous material.

21. The method of claim 18, wherein the step of introducing the porous material in the bed reactor comprises arranging the porous material in an assembly of several layers of a few hundred micrometers, preferably the layers of the porous material are separated by intermediate walls disposed within the bed reactor.
